(19) United States
(12) Patent Application Publication
(10) Pub. No.: US 2026/0021230 A1
(43) Pub. Date: Jan. 22, 2026

(54) SYSTEM AND METHOD FOR PRE-HEATING DIALYSATE USED IN DIALYSIS TREATMENT

(71) Applicants: Fresenius Medical Care Deutschland Gmbh, Bad Homburg (DE); Fresenius Medical Care Holdings, Inc., Waltham, MA (US)

(72) Inventors: Christian Schlaeper, Wehrheim (DE); Martin Crnkovich, Walnut Creek, CA (US); David Yuds, Hudson, NH (US)

(73) Assignees: Fresenius Medical Care Deutschland Gmbh, Bad Homburg (DE); Fresenius Medical Care Holdings, Inc., Waltham, MA (US)

(21) Appl. No.: 18/775,553

(22) Filed: Jul. 17, 2024

(51) Int. Cl. A61M 1/16; A61M 1/14; G16H 20/17; G16H 40/67

(52) U.S. Cl. CPC A61M 1/1607; A61M 1/154; G16H 20/17; G16H 40/67

(57) ABSTRACT

Systems and methods for pre-heating dialysate in a dialysis treatment system are disclosed. The system may include a dialysis machine including a pump to move dialysate through the dialysis machine, a dialysate heater to heat the dialysate, a control circuit operatively associated with the dialysate heater to activate and deactivate the dialysate heater, and a processing circuit. The processing circuit is configured to determine whether a trigger event has occurred, wherein whether the trigger event has occurred is determined based on a status of a dialysis patient assigned to the dialysis machine or a status of a dialysis clinic at which the dialysis machine is located. In response to the trigger event occurring, the processing circuit is configured to send a control signal to the control circuit to activate the dialysate heater and begin heating dialysate to a predetermined temperature.

### FIELD OF THE DISCLOSURE

The disclosure generally relates to dialysis machines, and more particularly, to a system and method for pre-heating dialysate fluid used in dialysis treatment to facilitate, for example, on-demand heating of the dialysate to avoid unnecessary heating and energy waste.

### BACKGROUND

Dialysis machines and/or systems are used in the treatment of renal disease. The two principal dialysis methods are hemodialysis (HD) and peritoneal dialysis (PD). During HD, the patient's blood is passed through a dialyzer while also passing dialysate through the dialyzer. A semi-permeable membrane in the dialyzer separates the blood from the dialysate within the dialyzer and allows diffusion and convection exchanges to take place between the dialysate and the blood stream. During PD, the patient's peritoneal cavity is periodically infused with dialysate or dialysis solution. The membranous lining of the patient's peritoneum acts as a natural semi-permeable membrane that allows diffusion and osmosis exchanges to take place between the solution and the blood stream.

Dialysis centers and clinics can have numerous dialysis machines that need to be prepared at the beginning of each treatment day. During this start-up sequence, for example, the HD machines draw acid and bicarbonate concentrates from central feed systems, heat reverse osmosis (RO) water from the water inlet, and proportionally mix the three streams together so the final product (dialysate) meets conductivity and temperature requirements before the dialyzer is even connected to the machine's hydraulics. Fresh dialysate is constantly being heated to match body temperature, even if this is long before a patient is connected to the machine. Depending on how long before the patient is ready, this may significantly waste electricity used to power the heater that heats the dialysate. Aggregated over 30 million HD treatments per year, a large carbon footprint is produced and presents a significant load on the electrical grid.

As such, there is a need for improved systems and methods for addressing some of the above-mentioned deficiencies.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In some embodiments, a method of pre-heating dialysate used in a dialysis treatment is disclosed. The method including determining, by a processing circuit, whether a trigger event has occurred, wherein whether the trigger event has occurred is determined based on a status of a dialysis patient assigned to the dialysis machine or a status of a dialysis clinic at which the dialysis machine is located, and in response to the trigger event occurring, sending, by the processing circuit, a control signal to a control circuit operatively associated with the dialysis machine to activate a dialysate heater and begin heating dialysate for the dialysis machine to a predetermined temperature.

In some embodiments, a system for pre-heating dialysate used in a dialysis treatment is disclosed. The system including a dialysis machine including a pump configured to move dialysate through the dialysis machine. The system also includes a dialysate heater configured to heat the dialysate and a control circuit operatively associated with the dialysate heater and configured to activate and deactivate the dialysate heater. The system further includes a processing circuit coupled to memory having executable instructions stored thereon, which when executed by the processing circuit, cause the processing circuit to determine whether a trigger event has occurred, wherein whether the trigger event has occurred is determined based on a status of a dialysis patient assigned to the dialysis machine or a status of a dialysis clinic at which the dialysis machine is located, and in response to the trigger event occurring, sending a control signal to the control circuit to activate the dialysate heater and begin heating dialysate for the dialysis machine to a predetermined temperature.

In another embodiment, a control system for a dialysis machine to pre-heat dialysate used in a dialysis treatment is disclosed. The control system including a control circuit operatively associated with a dialysate heater and configured to activate and deactivate the dialysate heater, the dialysate heater configured to heat dialysate for the dialysate machine. The control system further includes a processing circuit coupled to memory having executable instructions stored thereon, which when executed by the processing circuit, cause the processing circuit to determine whether a trigger event has occurred, wherein whether the trigger event has occurred is determined based on a status of a dialysis patient assigned to the dialysis machine or a status of a dialysis clinic at which the dialysis machine is located, and in response to the trigger event occurring, sending a control signal from the control circuit to the dialysate heater to activate the dialysate heater and begin heating dialysate to a predetermined temperature.

Non-transitory computer program products (i.e., physically embodied computer program products) are also described that store instructions, which, when executed by one or more data processors (i.e., processor circuit) of one or more computing systems, cause at least one data processor to perform operations herein. Similarly, computer systems are also described, which may include one or more data processors and memory coupled to the one or more data processors. The memory may temporarily or permanently store instructions that cause at least one processor to perform one or more of the operations described herein. In addition, methods can be implemented by one or more data processors, which are either within a single computing system or distributed among two or more computing systems. Such computing systems can be connected and can exchange data and/or commands or other instructions or the like via one or more connections, including but not limited to a connection over a network (e.g., the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE SEVERAL VIEWS OF THE DRAWINGS

By way of example, specific embodiments of the disclosed methods and devices will now be described, with reference to the accompanying drawings, in which:

FIG. 1 illustrates a block diagram of a system in accordance with one embodiment.

FIG. 2 illustrates a block diagram of a system in accordance with one embodiment.

FIG. 3 illustrates a block diagram of a system in accordance with one embodiment.

FIG. 4 illustrates a block diagram of a system in accordance with one embodiment.

FIG. 5 illustrates a block diagram of a system in accordance with one embodiment.

FIG. 6 illustrates a block diagram of a control system in accordance with one embodiment.

FIG. 7 illustrates a method in accordance with one embodiment.

It should be understood that the drawings are not necessarily to scale and that the disclosed embodiments are sometimes illustrated diagrammatically and in partial views. In certain instances, details which are not necessary for an understanding of the disclosed methods and devices or which render other details difficult to perceive may have been omitted. It should be further understood that this disclosure is not limited to the particular embodiments illustrated herein. In the drawings, like numbers refer to like elements throughout unless otherwise noted.

### DETAILED DESCRIPTION

With general reference to notations and nomenclature used herein, one or more portions of the detailed description which follows may be presented in terms of program procedures executed on a computer or network of computers. These procedural descriptions and representations are used by those skilled in the art to most effectively convey the substances of their work to others skilled in the art. A procedure is here, and generally, conceived to be a self-consistent sequence of operations leading to a desired result. These operations are those requiring physical manipulations of physical quantities. Usually, though not necessarily, these quantities take the form of electrical, magnetic, or optical signals capable of being stored, transferred, combined, compared, and otherwise manipulated. It proves convenient at times, principally for reasons of common usage, to refer to these signals as bits, values, elements, symbols, characters, terms, numbers, or the like. It should be noted, however, that all of these and similar terms are to be associated with the appropriate physical quantities and are merely convenient labels applied to those quantities.

Further, these manipulations are often referred to in terms, such as adding or comparing, which are commonly associated with mental operations performed by a human operator. However, no such capability of a human operator is necessary, or desirable in most cases, in any of the operations described herein that form part of one or more embodiments. Rather, these operations are machine operations. Useful machines for performing operations of various embodiments include digital computers as selectively activated or configured by a computer program stored within that is written in accordance with the teachings herein, and/or include apparatus specially constructed for the required purpose or a digital computer. Various embodiments also relate to apparatus or systems for performing these operations. These apparatuses may be specially constructed for the required purpose. The required structure for a variety of these machines will be apparent from the description given.

Embodiments of the present disclosure will now be described more fully hereinafter with reference to the accompanying drawings, in which several exemplary embodiments are shown. The subject matter of the present disclosure, however, may be embodied in many different forms and types of methods, systems, and devices for dialysis treatments and other potential medical devices and treatments, and should not be construed as limited to the embodiments set forth herein. Rather, these embodiments are provided so that this disclosure will be thorough and complete, and willfully convey the scope of the subject matter to those skilled in the art.

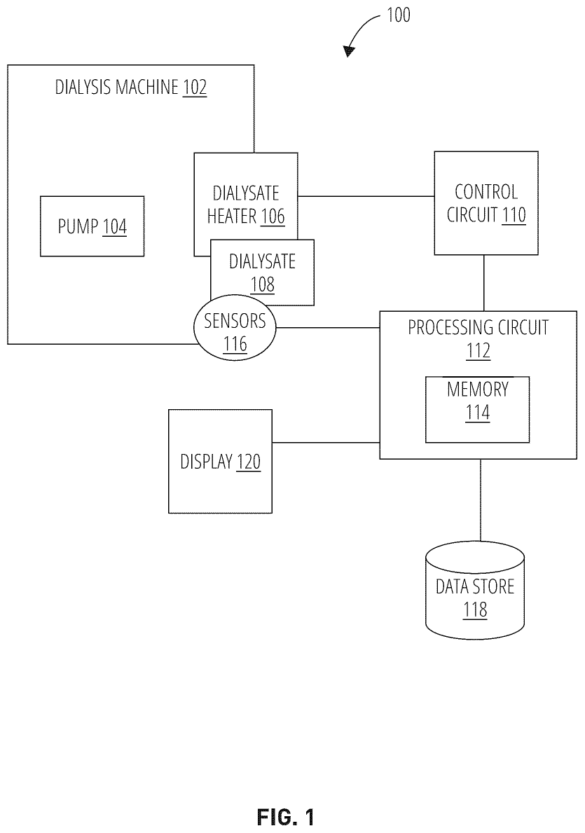

FIG. 1 is a block diagram illustrating a system 100 for pre-heating dialysate used in a dialysis treatment. The system 100 includes a dialysis machine 102. Those having ordinary skill in the art will understand that a dialysis machine 102 is a machine used to perform a dialysis treatment including, for example, removing toxins and wastes from a patient's bloodstream. Dialysis is typically used to treat patients who are experiencing kidney failure, kidney disease, or other condition causing their kidney function to be depleted.

During treatment, dialysate 108 is used by the dialysis machine 102 to remove toxins and wastes from the patient's blood. For example, the patient's blood may be pumped via tubing from the patient to a dialyzer. In use, the dialyzer may include a cartridge or series of tubes including a semi-permeable membrane (e.g., a high flux membrane) arranged and configured to remove toxins from the blood. Meanwhile, the dialysate is pumped through the dialyzer in the opposite direction as the blood. Within the dialyzer, the dialysate 108 interacts with the patient's blood to remove the toxins and wastes from the blood through diffusion of the wastes and toxins via the semi-permeable membrane.

Because the blood interacts with the dialysate 108, if the dialysate is not of the proper temperature (e.g., bodily temperature), heat transfer can occur between the blood and the dialysate (e.g., if the dialysate is cooler than the blood). As such, it is desirable in some cases to heat the dialysate before dialysis occurs to minimize any health-related effects of cooling the patient's blood. System 100 helps to pre-heat the dialysate to an appropriate temperature before the patient arrives for the dialysis treatment. The system 100, and other systems described herein, also provide mechanisms that save energy by not constantly running the dialysate heater 106 to keep the dialysate 108 at the appropriate temperature for an extended period of time. That is, the system 100 and other systems described herein begin pre-heating the dialysate 108 at a predetermined time in advance of the dialysis treatment to give the dialysate heater 106 adequate time to heat the dialysate 108 to the desired temperature at the required time (e.g., time approximate the commencement of the dialysis treatment (e.g., time the patient is connected to the dialysis machine 102)).

In some embodiments, the system 100 includes a dialysis machine 102 including a pump 104 configured to move dialysate 108 through the dialysis machine 102 and/or system. Although depicted in FIG. 1 as being integrated in the dialysis machine 102, the pump 104 can be located outside of and separate from the dialysis machine 102 with tubes connecting the pump 104 to the dialysis machine 102 for dialysate 108 to be moved through the dialysis machine 102 and/or system.

In some embodiments, the system 100 further includes a dialysate heater 106 to heat the dialysate 108. In some embodiments, the dialysate heater 106 can be integrated with the dialysis machine 102 and be located within an enclosure of the dialysis machine 102. In alternate embodiments, the dialysate heater 106 can be separate from the dialysis machine 102 and located outside of an enclosure thereof. In either configuration, the dialysate heater 106 is configured to heat the dialysate 108 to a predefined temperature. In some cases, the predefined temperature is approximately equal to a body temperature of a human. For example, the predefined temperature can be between 34 degrees Celsius (C) and 38° C. In another example, the predefined temperature to which the dialysate 108 can be heated is between 35.5° C. to 37° C. The dialysate heater 106 can include any suitable heating device now known or hereafter developed such as, for example, a heating element, a solar powered heating system, an electric resistance heater, radiant heater, furnace, boiler, or any suitable heating system.

In some embodiments, the system 100 further includes a control circuit 110 operatively associated with the dialysate heater 106 and configured to activate and deactivate the dialysate heater 106. For example, the control circuit 110 can be located outside the dialysis machine 102 or it can be integrated within the dialysis machine 102 and located within a housing thereof. The control circuit 110 can include a connector connecting it to the dialysate heater 106. The control circuit 110 is configured to operate the dialysate heater 106 (e.g., turn ON and OFF), for example, the control circuit 110 can control a flow of electrical current to the dialysate heater 106. To power ON the dialysate heater 106, the control circuit 110 directs a flow of electrical current to the dialysate heater 106, and to power OFF the dialysate heater 106, the control circuit 110 terminates the flow of electrical current to the dialysate heater 106. The control circuit 110 can include any suitable circuit components now known or hereafter developed to direct or terminate the flow of current to the dialysate heater 106. For example, the control circuit 110 can include a switch, a transistor, or any other suitable circuit component to direct or terminate the flow of electrical current.

The control circuit 110 can also include, or be in communication with, one or more temperature sensors (not shown), which may be configured to measure or determine a temperature of the dialysate heater 106 and/or dialysate 108. In use, the control circuit 110 can be configured to direct the flow of electrical current to the dialysate heater 106 based on one or more temperature readings of the sensors measuring the dialysate heater 106 and/or dialysate 108. If the measured temperature of the dialysate heater 106 and/or dialysate 108 is too hot, or exceeds a predetermined temperature (i.e., the desired bodily temperature), the control circuit 110 is configured to automatically turn the dialysate heater 106 OFF (i.e., terminate the flow of electrical current) in order to minimize damage to the dialysate 108 or injury to the patient. In some embodiments, the control circuit 110 can control the heat output of the dialysate heater 106 to maintain the temperature of the dialysate 108 to achieve the predetermined temperature discussed herein.

In some embodiments, the control circuit 110 can be separate from the dialysate heater 106 and connected thereto using one or more electrical connectors (e.g., one or more wires or one or more leads). In alternate embodiments, the control circuit 110 may be integrated with the dialysate heater 106 and connected directly thereto. For example, in some embodiments, both the dialysate heater 106 and the control circuit 110 can be collocated with each other on the dialysis machine 102 or within a housing of the dialysis machine 102. In alternate embodiments, the control circuit 110 can be integrated with the dialysis machine 102 and located within a housing of the dialysis machine 102 and the dialysate heater 106 can be located outside the dialysis machine 102, and connected thereto using tubes. In yet another alternate embodiment, the dialysate heater 106 may be integrated within the housing of the dialysis machine 102 and the control circuit 110 may be located outside of and separate from the dialysis machine 102. In use, any configuration of dialysis machine 102, dialysis heater 106, and control circuit 110 is envisioned and the present disclosure should not be limited to any particular configuration unless explicitly claimed.

The system 100 may further include a processing circuit 112 coupled to memory 114 having executable instructions stored thereon, which when executed by the processing circuit 112, causes the processing circuit 112 to perform various operations. In some embodiments, the processing circuit 112 may be collocated with both the dialysate heater 106 and the control circuit 110 on or within the dialysis machine 102, or the processing circuit 112 may be located separate from the dialysis machine 102, the dialysate heater 106, and/or the control circuit 110. In any event, the processing circuit 112 may be in communication with the control circuit 110 to exchange control signals therewith. In use, the control signals direct the control circuit 110 to control (e.g., turn ON) the flow of electrical current to the dialysate heater 106 or terminate (e.g., turn OFF) the flow of current thereto. That is, in response to the control circuit 110 receiving control signals from the processing circuit 112, the control circuit 110 is configured to either direct the flow of current to the dialysate heater 106 (e.g., in response to a “power ON” signal from the processing circuit 112) or terminate the flow of current to the dialysate heater 106 (e.g., in response to a “power OFF” signal from the processing circuit 112).

In some embodiments, the memory 114 includes executable instructions stored thereon that, when executed by the processing circuit 112, cause the processing circuit 112 to determine whether a trigger event has occurred. In some embodiments, whether the trigger event has occurred is determined based on a status of a dialysis patient assigned to the dialysis machine 102 or a status of a dialysis clinic at which the dialysis machine 102 is located. Possible trigger events, including triggers associated with the status of the dialysis patient and triggers associated with the dialysis clinic are provided in more detail below.

In response to the trigger event occurring or in response to the processing circuit 112 determining that the trigger event has occurred, the processing circuit 112 is configured to send a control signal to the control circuit 110 to activate the dialysate heater 106 and begin heating dialysate 108 for the dialysis machine 102 to the predetermined temperature discussed above.

In some embodiments, the system 100 may further include sensors 116 configured to measure the temperature of the dialysate 108. The sensors 116 may be in communication with the processing circuit 112. For example, the processing circuit 112 can be connected to the sensors 116 via a wireless connection or by a wired connection. In such an embodiment, the processing circuit 112 may be configured to poll the temperature sensors 116 and determine, from readings from the temperature sensors 116, a present temperature of the dialysate 108. In some examples, the temperature sensors 116 may be located in a tank holding the dialysate 108. In some alternate embodiments, the sensors 116 may be distributed throughout tubing for the dialysate 108. In some embodiments, the processing circuit 112 polls the sensors 116 and from the readings of the sensors 116 determine an average temperature of the dialysate 108. In some embodiments, the processing circuit 112 may receive a temperature reading from one sensor 116 in a tank holding the dialysate 108 and that is the temperature used by the processing circuit 112. By distributing the sensors 116, a more accurate picture of the temperature of the dialysate 108 can be gleaned by the processing circuit 112.

In some embodiments, the system 100 may further include a data store 118 having historical data stored thereon. The historical data can include historical data indicating how long the dialysate 108 requires to be heated by the dialysate heater 106 from a beginning temperature to the predetermined temperature. For example, the data store 118 may include data indicating how long the dialysate heater 106 has historically taken to heat the dialysate 108 from 22° C. to 37° C. The data store 118 can also include data on how long the dialysate heater 106 requires to heat the dialysate heater 106 from other beginning temperatures as well to the predetermined temperature of between 34° C. and 38° C. For example, in some embodiments, the data store 118 may include tables correlating time needed to heat the dialysate from the initial temperature to the predetermined temperature.

In use, the processing circuit 112 is configured to query the data store 118 to determine a historical amount of time the dialysate heater 106 has taken (e.g., needed time) to heat the dialysate from the present temperature of the dialysate, measured by the sensors 116, to the predetermined temperature. The processing circuit 112 is further configured to determine a future time at which the dialysis machine 102 will be used by the dialysis patient. In this case, the future time at which the dialysis machine 102 will be used by the dialysis patient and also the trigger event is determined based on the status of the dialysis clinic. For example, to determine the future time, the processing circuit 112 is configured to determine the future time based on one or more of the following statuses of the dialysis clinic: a patient schedule of the dialysis clinic; a patient workflow history at the dialysis clinic; and a current operational status of the dialysis clinic.

For example, the current temperature of the dialysate 108, as measured by the sensors 116 is measured to be 22° C. The historical heating up time for heating dialysate 108, that is at 22° C., to 37° C. has been determined by the processing circuit 112, based on an analysis of the historical data, was 15 minutes. Therefore, to heat the current dialysate 108 to 37° C., the processing circuit 112 will send the control signal to the control circuit 110 to direct current to the dialysate heater 106 15 minutes before the patient is supposed to start dialysis treatment. However, when the patient is supposed to start dialysis can be dependent upon the status of the dialysis clinic. For example, the patient schedule of the dialysis clinic might indicate that all of the dialysis machines 102 in the clinic will be in use when the patient is scheduled to arrive, so the processing circuit 112 will take the patient schedule of the dialysis clinic into account when determining when to send the control signal to the control circuit 110 to power ON the dialysate heater 106. The patient schedule of the dialysis clinic might also indicate other factors such as time of day and staffing concerns at the clinic. A patient workflow history at the dialysis clinic may also be consulted to determine how long the dialysis clinic has historically taken to complete treatment of a patient. A current operational status of the dialysis clinic may also inform the processing circuit 112 when the control signal should be sent. For example, if there is a power outage and only some of the dialysis machines 102 are able to be powered ON, this will impact the processing circuit 112 determination about when to send the control signal.

It should be noted that the example timeframes for heating the dialysate 108 (e.g., 15 minutes) were provided for illustrative purposes only and should not be construed as limiting the present disclosure. The warm-up time can be any time between a few seconds (e.g., 1-3 seconds) to 30 minutes, or more.

Once the processing circuit 112 determines how long the dialysate heater 106 has historically taken to heat the dialysate 108 from the present temperature to the predetermined temperature, and the processing circuit 112 has considered the status of the dialysis clinic, the processing circuit 112 is configured to send the control signal to the control circuit 110 to activate the dialysate heater 106 prior to the future time (e.g., when the patient is to receive dialysis) by an amount equal to the historical amount of time plus any additional time based on the considerations of the status of the dialysis clinic. For example, if it takes 15 minutes to heat the dialysate 108 from 22° C. to 37° C. (e.g., the predetermine temperature), but the clinic has a 15 minute delay, the processing circuit 112 may wait an additional 15 minutes to account for the clinic's delay before sending the control signal.

In some embodiments, instead of automatically sending the control signal, the processing circuit 112 can be configured to display a timer on a display 120 indicating how long it is expected to take for the dialysate 108 to heat up to the predetermined temperature and/or conductivity so a technician knows when to activate the dialysate heater 106 (e.g., via a button or switch) in advance of the patient's treatment.

In some embodiments, the dialysis machine 102 is a hemodialysis machine. In other embodiments, the dialysis machine 102 is a peritoneal dialysis machine.

In some cases, the data store 118 can include historical data for patients. For example, the data store 118 can include medical history data. In some cases, the predetermined temperature discussed above can be determined by the processing circuit 112 based on an analysis of medical history data of the dialysis patient, the medical history data including at least body temperature data of the dialysis patient. For example, if the patient's medical history demonstrates that the dialysis patient has an average historical body temperature of 36.5° C., the processing circuit 112 and control circuit 110 can operate to control the dialysate heater 106 to maintain the dialysate 108 at a temperature of 36.5° C. The processing circuit 112 can keep the dialysate 108 at the predetermined temperature using the sensors 116 to monitor the current temperature of the dialysate 108 and power cycling the dialysate heater 106 as needed to maintain the predetermined temperature.

In some cases, elements of the dialysis machine 102 may also cause delay in how long it takes the dialysate 108 to heat up to the predetermined temperature discussed above. For example, the warm-up time period may take longer because of a maintenance issue with the dialysis machine 102 (e.g., a deaeration pump). In such a case, the processing circuit 112 may cause a notification to be displayed on the display 120 for a technician to review the hydraulic and other components of the dialysis machine 102.

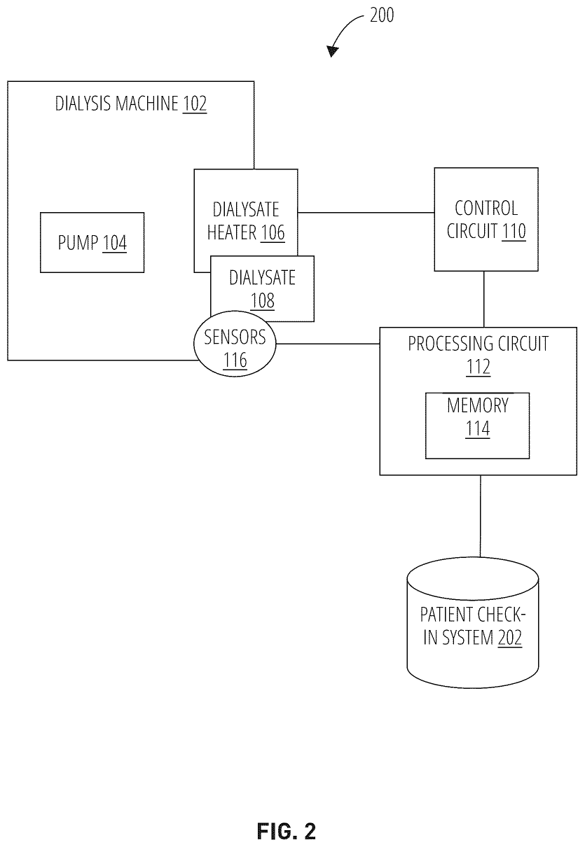

FIG. 2 is a block diagram illustrating another example of a system 200 for pre-heating dialysate used in a dialysis treatment. As shown in FIG. 2, the system 200 is similar to system 100, however, in this example, whether the trigger event has occurred is determined based on the status of the dialysis patient, in addition to or instead of, the status of the dialysis clinic as discussed above.

In this example embodiment, the status of the dialysis patient may relate to, for example, the dialysis patient checking in to the dialysis clinic for their dialysis treatment. For example, the system 200 can include a patient check-in system 202, such a software service that allows the dialysis patients to check in to their appointment when they arrive at the dialysis clinic. In use, the processing circuit 112 may be in communication with the patient check-in system 202, and the trigger event can include the patient checking in at the patient check-in system 202. In such an example, the processing circuit 112 may receive a check-in notification from the patient check-in system 202 that dialysis patient assigned to the dialysis machine 102 has checked in to the dialysis clinic.

In response to the processing circuit 112 receiving the check-in notification, the processing circuit 112 may send the control signal a predetermined amount of time after receiving the check-in notification, the predetermined amount of time being determined based at least on how long after checking in, it takes the dialysis patient to begin receiving dialysis. When the control signal is sent can also be based on the current temperature of the dialysate 108 as measured by the sensors 116. To make this determination, the processing circuit 112 can query the data store 118 from FIG. 1 and look at historical heat-up times as described above.

The predetermined amount of time can be determined based on the current temperature of the dialysate 108, the predetermined temperature the dialysate 108 is to be heated to, and a time it takes (e.g., an average time, a median time, etc.) for the dialysis patient to start dialysis after they have checked in. For example, if the dialysis patient has just checked in, and it takes the dialysate heater 106 15 minutes to heat the dialysate 108 from 22° C. to 37° C. and it takes the patient 20 minutes to go from check-in to starting dialysis treatment, the processing circuit 112 will take these time ranges into account and wait approximately 5 minutes to send the control signal to power on the dialysate heater 106 so that the dialysate heater 106 is unnecessarily heating the dialysate 108 and wasting energy.

In addition, the processing circuit 112 can take into account the dialysis clinic parameters discussed above with respect to FIG. 1 when determining when to send the control signal.

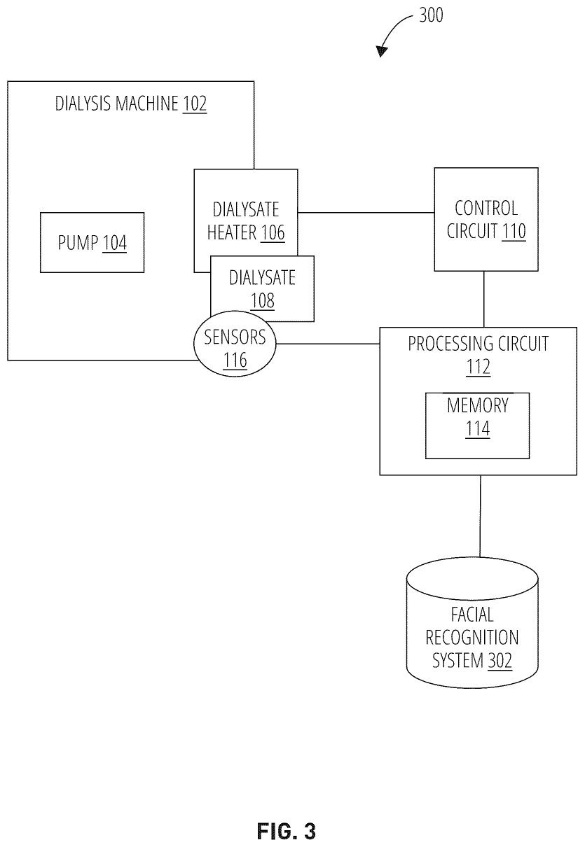

FIG. 3 is a block diagram illustrating another system 300 for pre-heating dialysate used in a dialysis treatment. As shown in FIG. 3, the system 300 is similar to FIG. 2 above whereby, whether the trigger event has occurred is determined based on the status of the dialysis patient, in addition to or instead of, the status of the dialysis clinic as discussed above. In FIG. 3, the system 300 may include, for example, a facial recognition system 302 configured to scan faces of people entering the dialysis clinic. In some embodiments, the facial recognition system 302 includes one or more cameras located adjacent to an entrance of the dialysis clinic and software to recognize facial features of the people entering the clinic. Alternatively, the one or more cameras can be located at any suitable location (e.g., entryway, entry door, side door, clinic office door, etc.) such that facial recognition can be performed.

The software may include a database of pictures or features of known patients at the clinic (e.g., captured when the patient enrolled at the clinic), and the software can detect the presence of the patient based on the cameras capturing pictures of the patient entering the clinic and comparing features of people entering the clinic with those features in the database. Once a patient has entered the clinic or any other area near the dialysis machine 102, the facial recognition system 302 will detect their presence via the facial recognition software and then send a message to the processing circuit 112 that the patient has arrived.

In some embodiments, the processing circuit 112 receives the message from the facial recognition system 302 that indicates that the dialysis patient has entered an area near the dialysis machine 102 (e.g., the clinic, a waiting room, or any other location near the dialysis machine 102 that indicates that the dialysis patient will be receiving dialysis treatment soon). This is the trigger event discussed above. In response to the processing circuit 112 receiving the message from the facial recognition system 302 that the patient has arrived, the processing circuit 112 sends the control signal a predetermined amount of time after receiving the message, the predetermined amount of time being determined based at least on a distance between the area and the dialysis machine.

For example, the facial recognition system 302 may detect that the patient has entered the clinic, but the clinic is very large or it may take some time to get to the area where dialysis occurs, or for the patient to check-in. As such, the processing circuit 112 takes this time into consideration to determine when to send the control signal. In some embodiments, other determinations regarding when the processing circuit 112 should send the control signal discussed above, may also be utilized by the processing circuit 112 in this embodiment as well.

Using the example above, wherein the dialysis patient has just been recognized by the facial recognition system 302, and it takes the dialysate heater 106 15 minutes to heat the dialysate 108 from 22° C. to 37° C. and it takes the patient 5 minutes to get from the area the facial recognition system 302 detected the patient and another 15 minutes to complete check-in procedures and then proceed to starting dialysis treatment, the processing circuit 112 will take these time ranges into account and wait approximately 5 minutes to send the control signal to power ON the dialysate heater 106 so that the dialysate heater 106 is not unnecessarily heating the dialysate 108 and wasting energy.

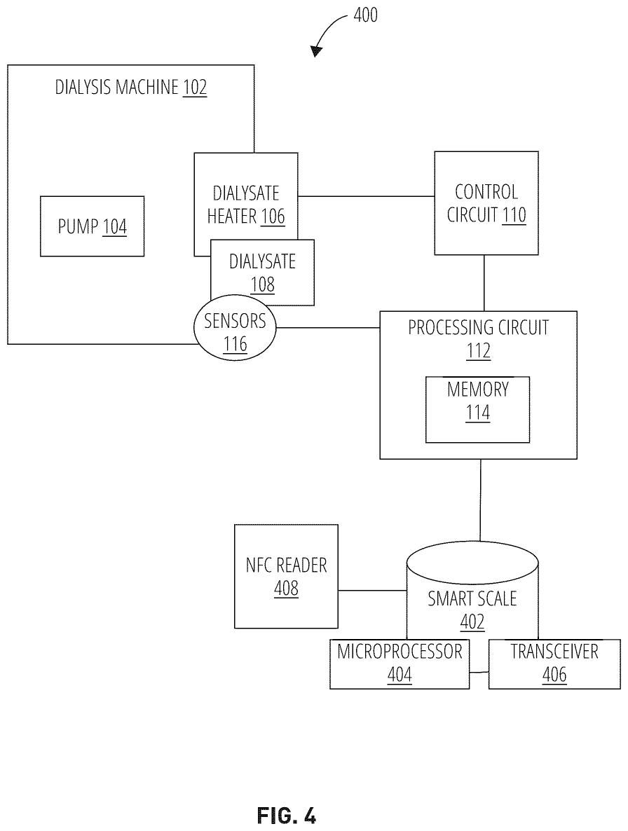

FIG. 4 is a block diagram illustrating another system 400 for pre-heating dialysate used in a dialysis treatment. As shown in FIG. 4, the system 400 is similar to FIG. 2 and FIG. 3 above wherein, whether the trigger event has occurred is determined based on the status of the dialysis patient, in addition to or instead of, the status of the dialysis clinic as discussed above. In this embodiment, the system 400 may include a smart scale 402 including a microprocessor 404 and a transceiver 406 for communicating with the processing circuit 112.

In some embodiments, the smart scale 402 is a scale to measure a weight or mass of a patient. For example, the smart scale 402 can include a scale for weighing patients that is connected to the Internet or has some other network connection (e.g., wired or wireless connection) to the processing circuit 112. In use, the smart scale 402 communicates via the network connection with the processing circuit 112 via the microprocessor 404 and transceiver 406. For example, the smart scale 402 may include a near field communication (NFC) reader 408 for reading an NFC enabled device associated with the patient, or some other suitable wireless communication reader. For example, the patient may have an NFC enabled card (e.g., NFC or radio frequency identification (RFID) card) or use their phone to tap the NFC reader 408 and indicate their presence to the smart scale 402. Alternatively, the NFC reader 408 can be tapped by a nurse or other healthcare provider using their badge and some other mechanism of identifying the dialysis patient as the patient being weighed. For example, a patient barcode or patient number may be entered into a user interface associated with the smart scale 402 indicating that the dialysis patient is the one being weighed. The NFC reader 408 can also include an RFID reader.

In any case, the smart scale 402 determines the dialysis patient being weighed thereon, for example, based on a biometric reading (e.g., using a biometric scanner device not shown) of the dialysis patient or a near field communication (NFC) reading of an NFC enabled device associated with the dialysis patient at the smart scale 402, or any other suitable mechanism now known or hereafter developed for identifying the patient being weighed on the smart scale 402. After the smart scale 402 makes the determination, the smart scale 402 sends a message to the processing circuit 112 that the dialysis patient is being weighed thereon based on the determination. In some cases, the smart scale 402 sends, as part of the message, data indicating a weight of the dialysis patient for use by the dialysis machine 102 for preparing components thereof for the dialysis patient. The processing circuit 112 may further send the control signal to the control circuit 110 in response to receiving the message from the smart scale 402.

The smart scale 402 weighing the patient is the trigger event discussed above. In this example, the processing circuit 112 may immediately send the control signal upon receiving the message that the patient is being weighed. Alternatively, the processing circuit 112 can take into account some of the other factors considered above to determine whether to delay sending the control signal. For example, the processing circuit 112 may delay sending the control signal based on historical data indicating how long it has historically taken to warm up the dialysate 108 or how long it takes the patient to transition from being weighed at the smart scale 402 to being treated at the dialysis machine 102. In addition, the system 400 may also take into account the factors involving the clinic status.

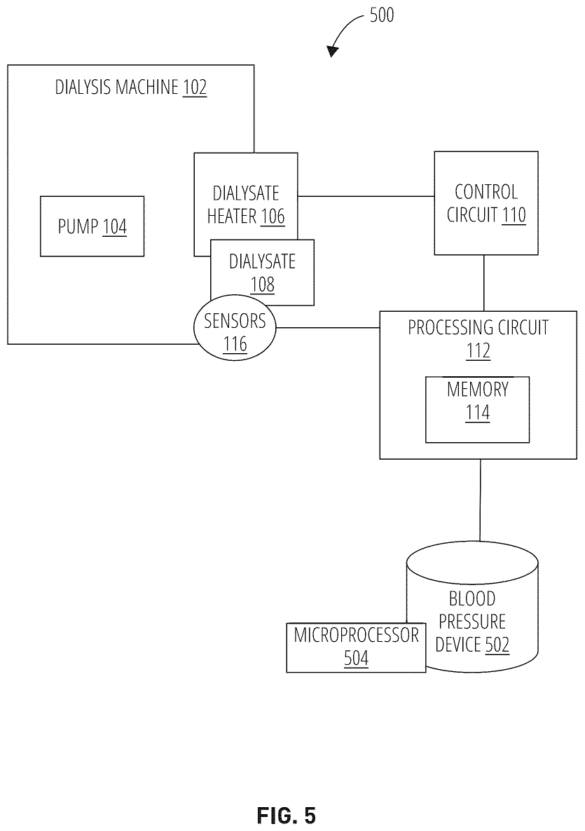

FIG. 5 is a block diagram illustrating another system 500 for pre-heating dialysate used in a dialysis treatment. As shown in FIG. 5, the system 500 is similar to system 400 above, however, instead of a smart scale 402, the system 500 includes a blood pressure device 502, in some embodiments, having a microprocessor 504 integrated therewith. In use, the blood pressure device 502 measures a blood pressure of the dialysis patient. In some cases, during an ordinary treatment flow, the blood pressure of the patient is taken just before the dialysis patient is connected to the dialysis machine 102 for dialysis treatment. For example, the patient may be weighed, then their blood pressure taken, then shortly thereafter, connected to the dialysis machine 102 for treatment. In some cases, the blood pressure device 502 may be a separate machine from the dialysis machine 102. In other embodiments, the dialysis machine 102 may have a blood pressure device 502 integrated therewith. That is, the patient's blood pressure may be taken separately from the dialysis machine 102 (e.g., at a large distance from the dialysis machine 102) or their blood pressure can be captured at the dialysis machine 102.

In connection with this embodiment, whether the trigger event has occurred is determined based on the status of the dialysis patient and the trigger event is the measurement of the patient's blood pressure. As discussed above, the blood pressure device 502 may include a microprocessor 504 to send a message to the processing circuit 112 that the patient's blood pressure is being measured. The blood pressure device 502 can determine that the patient is the one whose blood pressure is being measured based on similar features discussed above with respect to the smart scale 402 (e.g., NFC reader, biometric scanner, patient barcode, etc.). In this way, the blood pressure device 502 can detect that the patient is the one whose blood pressure is being measured and then send the message indicating that to the processing circuit 112.

In some embodiments, the processing circuit 112 may further receive the message from the blood pressure device 502 or the microprocessor 504 associated therewith that the dialysis patient is having or has had their blood pressure measured by the blood pressure device 502. In response to receiving the message from the blood pressure device 502, or the microprocessor 504 associated therewith, the processing circuit 112 sends the control signal to the control circuit 110 to begin heating the dialysate.

In this embodiment, the processing circuit 112 may immediately send the control signal upon receiving the message that the patient is having their blood pressure measured. Alternatively, the processing circuit 112 can take into account some of the other factors considered above to determine whether to delay sending the control signal. For example, the processing circuit 112 may delay sending the control signal based on historical data indicating how long it has historically taken to heat the dialysate 108 or how long it takes the patient to transition from having their blood pressure measured at the blood pressure device 502 to being treated at the dialysis machine 102. In addition, the system 500 may also take into account the factors involving the clinic status.

In some embodiments, the control system 600 may further include generating concentrates from a connected BiBag® disposable, which would save time due to its known pH and expected conductivity. As such, the dialysate 108 may be heated to the predetermined temperature and conductivity just as the clinician finishes making the blood access connections.

Furthermore, additional savings measures for clinics running multiple shifts on the same machines could more efficiently use the post-treatment heated BiBag® disposable. Because the systems described herein can access and analyze the clinic's schedule, and they can calculate the remaining concentrate in the BiBag® disposable at the end of the treatment, in some embodiments, the systems described herein may prompt the clinician to leave the previous treatment's BiBag® on the machine and automatically run the next self-test as the patient waits for clotting after treatment. The BiBag® would be efficiently used up to ensure the machine is tested and ready for the next treatment and then prompt the clinician to connect a fresh, full BiBag® disposable just in time for the next patient.

Each of these implementations would also add great value to the self-care clinic model with any of the prompts delivered through the patient's smartphone or smartwatch. By keeping the patient on task, a green self-care clinic can run much more efficiently, with as little energy wasted as possible.

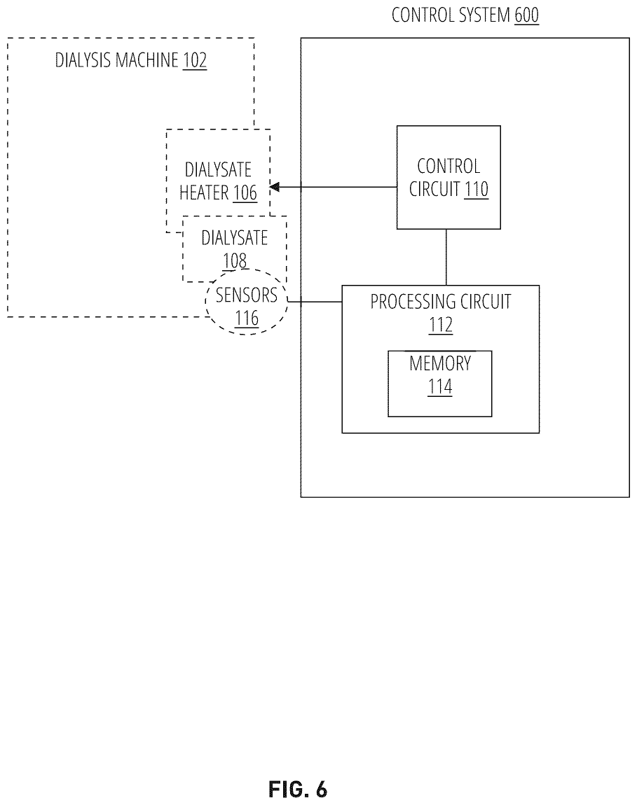

FIG. 6 is a block diagram illustrating a control system 600 for pre-heating dialysate used in a dialysis treatment. In some embodiments, the control system 600 utilizes a dialysate heater 106 associated with a dialysis machine 102 to pre-heat the dialysate 108 used in a dialysis treatment. The control system 600 includes a control circuit 110 operatively associated with the dialysate heater 106 to activate and deactivate the dialysate heater 106, the dialysate heater 106 configured to heat the dialysate. The control circuit 110 can include a similar or the same control circuit 110 discussed above with respect to FIG. 1.

The control system 600 further includes a processing circuit 112 coupled to memory 114 having executable instructions stored thereon, which when executed by the processing circuit, cause the processing circuit to perform various operations. For example, in some embodiments, executing the instructions causes the processing circuit 112 to determine whether a trigger event has occurred, wherein whether the trigger event has occurred is determined based on a status of a dialysis patient assigned to the dialysis machine 102 or a status of a dialysis clinic at which the dialysis machine 102 is located.

In some embodiments, executing the instructions further causes the processing circuit 112 to, in response to the trigger event occurring, send a control signal to the control circuit 110 to activate the dialysate heater 106 and begin heating the dialysate 108 to a predetermined temperature. The control signal can be any of the control signals discussed above and the predetermined temperature can be any of the predetermined temperatures discussed above.

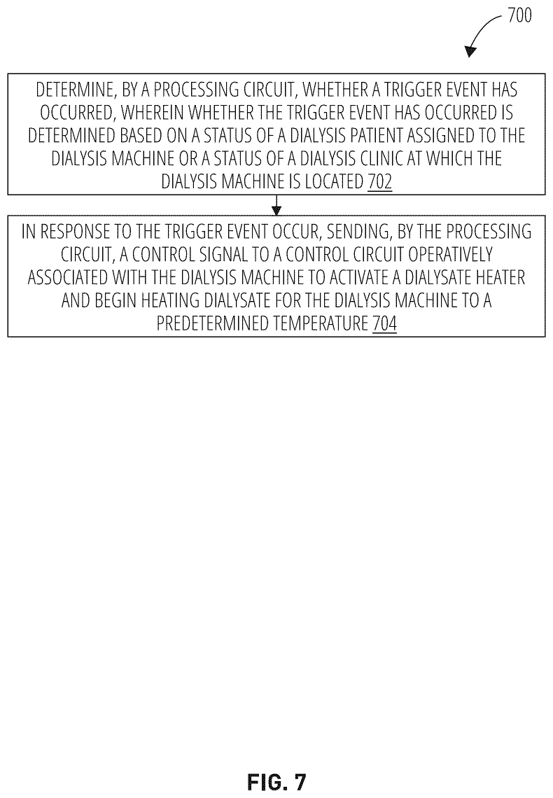

FIG. 7 is a flow chart illustrating various operations of a method 700 for pre-heating dialysate fluid used in dialysis treatment. As shown at block 702, the method 700 includes determining, by a processing circuit, whether a trigger event has occurred, wherein whether the trigger event has occurred is determined based on a status of a dialysis patient assigned to the dialysis machine or a status of a dialysis clinic at which the dialysis machine is located. As shown at block 704, the method 700 includes, in response to the trigger event occurs, sending, by the processing circuit, a control signal to a control circuit operatively associated with the dialysis machine to activate a dialysate heater and begin heating dialysate for the dialysis machine to a predetermined temperature.

In some embodiments, whether the trigger event has occurred is determined based on the status of the dialysis clinic, and determining that the trigger event has occurred includes: the processing circuit polling temperature sensors that measure temperature of the dialysate, and determining, from the temperature sensors, a present temperature of the dialysate; the processing circuit querying a data store, having historical data stored thereon, to determine a historical amount of time the dialysate heater has taken to heat the dialysate from the present temperature of the dialysate to the predetermined temperature; determining, by the processing circuit, a future time at which the dialysis machine will be used by the dialysis patient; and the processing circuit sending the control signal to the control circuit to activate the dialysate heater prior to the future time by an amount equal to the historical amount of time.

In some further embodiments, determining the future time at which the dialysis machine will be used by the dialysis patient includes the processing circuit determining the future time based on one or more of the following statuses of the dialysis clinic: a patient schedule of the dialysis clinic; a patient workflow history at the dialysis clinic; and a current operational status of the dialysis clinic.

In some embodiments, whether the trigger event has occurred is determined based on the status of the dialysis patient, and determining that the trigger event has occurred includes: receiving, by the processing circuit, a check-in notification that dialysis patient assigned to the dialysis machine has checked in to the dialysis clinic; and wherein sending the control signal to the control circuit to activate the dialysate heater includes sending the control signal a predetermined amount of time after receiving the check-in notification, the predetermined amount of time being determined based at least on how long after checking in it takes the dialysis patient to begin receiving dialysis.

In some embodiments, whether the trigger event has occurred is determined based on the status of the dialysis patient, and determining that the trigger event has occurred includes: receiving, by the processing circuit, a message from a facial recognition system that indicates that dialysis patient has entered an area near the dialysis machine; and wherein sending the control signal to the control circuit to activate the dialysate heater includes sending the control signal a predetermined amount of time after receiving the message, the predetermined amount of time being determined based at least on a distance between the area and the dialysis machine.

In some embodiments, whether the trigger event has occurred is determined based on the status of the dialysis patient, and determining that the trigger event has occurred includes: receiving, by the processing circuit from a smart scale including a microprocessor and a transceiver for communicating with the processing circuit, a message that indicates that dialysis patient assigned to the dialysis machine has been weighed by the smart scale; and the method further includes: determining, by the smart scale, that the dialysis patient is being weighed at the smart scale based on a biometric reading of the dialysis patient or a near field communication (NFC) reading of an NFC enabled device associated with the dialysis patient; sending, by the smart scale, the message based on the determination that the dialysis patient is being weighed; and sending, by the smart scale as part of the message, data indicating a weight of the dialysis patient for use by the dialysis machine for preparing components of the dialysis machine for the dialysis patient.

In some embodiments, whether the trigger event has occurred is determined based on the status of the dialysis patient, and determining that the trigger event has occurred includes receiving, by the processing circuit, a message from a blood pressure device that the dialysis patient assigned to the dialysis machine is having or has had their blood pressure measured by the blood pressure device.

In some embodiments, the dialysis machine includes an integrated blood pressure checking device and determining that the trigger event has occurred includes detecting that an activation switch of the blood pressure checking device has been activated and the dialysis patient assigned to the dialysis machine is having their blood pressure measured by the blood pressure checking device.

In some embodiments, the method further includes determining, by the processing circuitry, the predetermined temperature based on an analysis of medical history data of the dialysis patient, the medical history data including at least body temperature data of the dialysis patient.

For some embodiments, the dialysis machines can produce saline online, known as substitution fluid, the pre-heating procedures described above would have an additional technical challenge because the substitution fluid system needs intermittent fluid flow through the bloodline of 25-50 ml/minute (ml/min). As the substitution fluid goes through the dialyzer, these example dialysis machines would need to synchronize to run dialysate flow at 25 ml/min. This could be done using Hall effect sensors on each of the pumps as long as the system knows which bloodlines are connected to perform the calculation to synchronize. Instead of relying on the operator to intervene with scanning a quick response (QR) code on the bloodlines or manually entering the product number, the example systems described herein could intelligently deduce it when priming the bloodlines with substitution fluid and running dialysate flow with a set ultrafiltration (UF) during a self-test: intermittently running the blood pump and corresponding it to the expected UF volume would allow the system to precisely calculate the bloodline volume, whether standard, pediatric, or even a competitor's bloodlines, by comparing the results to a look-up table in the computer memory.

Some additional considerations to run the heater in the most efficient manner when producing substitution fluid are provided below. If the substitution fluid in the bloodlines is not constantly changed, a milky solution forms in the bloodlines-there must be some sort of fluid moving through bloodlines to prevent this (e.g., fluid flow provided by one or more pumps); however, it doesn't have to be constant. In some cases, the one or more pumps are operated such that the substitution fluid is provided at 30-60 ml/min but the machine could run intermittently fast and slow, creating positive flow and reducing clotting at dialyzer. The positive flow is also crucial at other times for preventing backflow during priming or if the lines need to be re-primed, therefore the lines must remain in a protected state of positive pressure.

The balancing chamber system of the hydraulics is excellent, but due to the nature of the diaphragm movements, the temperature and flow have spikes with the highest volume of the warmest fluid at the beginning of the stroke. This indicates that waves much be translated into periodic flow to maintain the highest efficiency at the lowest power. This can be accomplished using an additional insulated chamber inline which receives the flow and meters it out in a constant stream at a constant temperature.

Since no accuracy is needed when a patient is not on machine, the “cold” machine can run slowly with just enough momentum to keep fluid from stagnating.

When the time comes to ramp up the temperature and tighten up accuracy, the dialysate solution will be higher temperature at the beginning, but this heat can radiate through the system and equilibrate by the time the machine must be ready to connect to the patient. The pumps should run slow at first to build up pressure to reach the pressure sensors and then, when pressure has stabilized, they can run much faster. During this time, the substitution fluid pump must be linked to the stroke of the balancing chamber; this will allow us to create substitution fluid at the optimal temperature.

The systems and methods described herein have been explained in connection with dialysis machines having a particular configuration. It is contemplated that the systems described herein may be used with dialysis machines having other configurations, for example, different types of dialysis machines and/or dialysis machines having dialysate heaters in other configurations. The systems described herein may be used with any appropriate dialysis machine.

Some embodiments of the disclosed system may be implemented, for example, using a storage medium, a computer-readable medium or an article of manufacture which may store an instruction or a set of instructions that, when executed by a machine (e.g., processor, processing circuit, or microcontroller), may cause the machine to perform a method and/or operations in accordance with embodiments of the disclosure. In addition, a server or database server may include machine readable media configured to store machine executable program instructions. Such a machine may include, for example, any suitable processing platform, computing platform, computing device, processing device, computing system, processing system, computer, processor, or the like, and may be implemented using any suitable combination of hardware, software, firmware, or a combination thereof and utilized in systems, subsystems, components, or sub-components thereof. The computer-readable medium or article may include, for example, any suitable type of memory unit, memory device, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, and the like, implemented using any suitable high-level, low-level, object-oriented, visual, compiled and/or interpreted programming language.

As used herein, an element or operation recited in the singular and proceeded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the recited features.

While the systems and techniques described herein for pre-heating dialysate for a dialysis machine have been largely explained with reference to a dialysis machine, in particular, a hemodialysis machine, the systems and techniques described for pre-heating dialysate may be used in connection with other types of medical treatment systems and/or machines, such as a peritoneal machine or other medical treatment device involving medical fluids. In some implementations, the dialysis machine may be configured for use in a dialysis clinic or a patient's home (e.g., a home dialysis machine). The home dialysis machine can take the form of a peritoneal dialysis machine or a home hemodialysis machine.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A method of pre-heating dialysate used in a dialysis treatment utilizing a dialysis machine, the method comprising:
determining, by a processing circuit, whether a trigger event has occurred, wherein whether the trigger event has occurred is determined based on a status of a dialysis patient assigned to the dialysis machine or a status of a dialysis clinic at which the dialysis machine is located; and
in response to the trigger event occurring, sending, by the processing circuit, a control signal to a control circuit operatively associated with the dialysis machine to activate a dialysate heater and begin heating dialysate for the dialysis machine to a predetermined temperature.

2. The method of claim 1, wherein whether the trigger event has occurred is determined based on the status of the dialysis clinic, and determining that the trigger event has occurred comprises:
the processing circuit polling temperature sensors that measure temperature of the dialysate, and determining, from the temperature sensors, a present temperature of the dialysate;
the processing circuit querying a data store, having historical data stored thereon, to determine a historical amount of time the dialysate heater has taken to heat the dialysate from the present temperature of the dialysate to the predetermined temperature;
determining, by the processing circuit, a future time at which the dialysis machine will be used by the dialysis patient; and
the processing circuit sending the control signal to the control circuit to activate the dialysate heater prior to the future time by an amount equal to the historical amount of time.

3. The method of claim 2, wherein determining the future time at which the dialysis machine will be used by the dialysis patient includes the processing circuit determining the future time based on one or more of the following statuses of the dialysis clinic:
a patient schedule of the dialysis clinic;
a patient workflow history at the dialysis clinic; and
a current operational status of the dialysis clinic.

4. The method of claim 1, wherein whether the trigger event has occurred is determined based on the status of the dialysis patient, and determining that the trigger event has occurred comprises:
receiving, by the processing circuit, a check-in notification that dialysis patient assigned to the dialysis machine has checked in to the dialysis clinic; and
wherein sending the control signal to the control circuit to activate the dialysate heater comprises sending the control signal a predetermined amount of time after receiving the check-in notification, the predetermined amount of time being determined based at least on how long after checking in it takes the dialysis patient to begin receiving dialysis.

5. The method of claim 1, wherein whether the trigger event has occurred is determined based on the status of the dialysis patient, and determining that the trigger event has occurred comprises:
receiving, by the processing circuit, a message from a facial recognition system that indicates that dialysis patient has entered an area near the dialysis machine; and
wherein sending the control signal to the control circuit to activate the dialysate heater comprises sending the control signal a predetermined amount of time after receiving the message, the predetermined amount of time being determined based at least on a distance between the area and the dialysis machine.

6. The method of claim 1, wherein whether the trigger event has occurred is determined based on the status of the dialysis patient, and determining that the trigger event has occurred comprises:
receiving, by the processing circuit from a smart scale comprising a microprocessor and a transceiver for communicating with the processing circuit, a message that indicates that dialysis patient assigned to the dialysis machine has been weighed by the smart scale; and
wherein the method further comprises:
determining, by the smart scale, that the dialysis patient is being weighed at the smart scale based on a biometric reading of the dialysis patient or a near field communication (NFC) reading of an NFC enabled device associated with the dialysis patient;
sending, by the smart scale, the message based on the determination that the dialysis patient is being weighed; and
sending, by the smart scale as part of the message, data indicating a weight of the dialysis patient for use by the dialysis machine for preparing components of the dialysis machine for the dialysis patient.

7. The method of claim 1, wherein whether the trigger event has occurred is determined based on the status of the dialysis patient, and determining that the trigger event has occurred comprises receiving, by the processing circuit, a message from a blood pressure device that the dialysis patient assigned to the dialysis machine is having or has had their blood pressure measured by the blood pressure device; or
wherein the dialysis machine comprises an integrated blood pressure checking device and wherein determining that the trigger event has occurred includes detecting that an activation switch of the blood pressure checking device has been activated and the dialysis patient assigned to the dialysis machine is having their blood pressure measured by the blood pressure checking device.

8. The method of claim 1, wherein the dialysis machine is a hemodialysis machine.

9. The method of claim 1, the method further comprises determining, by the processing circuitry, the predetermined temperature based on an analysis of medical history data of the dialysis patient, the medical history data including at least body temperature data of the dialysis patient.

10. A system for pre-heating dialysate used in a dialysis treatment utilizing a dialysis machine, the system comprising:
a dialysis machine comprising a pump configured to move dialysate through the dialysis machine;
a dialysate heater configured to heat the dialysate;
a control circuit operatively associated with the dialysate heater and configured to activate and deactivate the dialysate heater; and
a processing circuit coupled to memory having executable instructions stored thereon, which when executed by the processing circuit, cause the processing circuit to:
determine whether a trigger event has occurred, wherein whether the trigger event has occurred is determined based on a status of a dialysis patient assigned to the dialysis machine or a status of a dialysis clinic at which the dialysis machine is located; and
in response to the trigger event occurring, send a control signal to the control circuit to activate the dialysate heater and begin heating dialysate for the dialysis machine to a predetermined temperature.

11. The system of claim 10, wherein whether the trigger event has occurred is determined based on the status of the dialysis clinic, and the system further comprises:
sensors that measure a temperature of the dialysate;
wherein the processing circuit is further to:
poll the temperature sensors and determine, from the temperature sensors, a present temperature of the dialysate;
query a data store, having historical data stored thereon, to determine a historical amount of time the dialysate heater has taken to heat the dialysate from the present temperature of the dialysate to the predetermined temperature;
determine a future time at which the dialysis machine will be used by the dialysis patient; and
send the control signal to the control circuit to activate the dialysate heater prior to the future time by an amount equal to the historical amount of time.

12. The system of claim 11, wherein determining the future time at which the dialysis machine will be used by the dialysis patient includes the processing circuit to determine the future time based on one or more of the following statuses of the dialysis clinic:
a patient schedule of the dialysis clinic;
a patient workflow history at the dialysis clinic; and
a current operational status of the dialysis clinic.

13. The system of claim 10, wherein whether the trigger event has occurred is determined based on the status of the dialysis patient, and the processing circuit is further to:
receive a check-in notification that dialysis patient assigned to the dialysis machine has checked in to the dialysis clinic; and
send the control signal a predetermined amount of time after receiving the check-in notification, the predetermined amount of time being determined based at least on how long after checking in it takes the dialysis patient to begin receiving dialysis.

14. The system of claim 10, wherein whether the trigger event has occurred is determined based on the status of the dialysis patient, and the processing circuit is further to:
receive a message from a facial recognition system that indicates that the dialysis patient has entered an area near the dialysis machine; and
send the control signal a predetermined amount of time after receiving the message, the predetermined amount of time being determined based at least on a distance between the area and the dialysis machine.

15. The system of claim 10, wherein whether the trigger event has occurred is determined based on the status of the dialysis patient, wherein the system further comprises:
a smart scale comprising a microprocessor and a transceiver for communicating with the processing circuit, wherein the smart scale is to:
make a determination that the dialysis patient is being weighed thereon based on a biometric reading of the dialysis patient or a near field communication (NFC) reading of an NFC enabled device associated with the dialysis patient at the smart scale;
send a message to the processing circuit that the dialysis patient is being weighed thereon based on the determination; and
send, as part of the message, data indicating a weight of the dialysis patient for use by the dialysis machine for preparing components of the dialysis machine for the dialysis patient
wherein the processing circuit is further to send the control signal to the control circuit in response to receiving the message from the smart scale.

16. The system of claim 10, wherein whether the trigger event has occurred is determined based on the status of the dialysis patient, and wherein the system further comprises:
a blood pressure device, separate from or integrated with the dialysis machine, to measure a blood pressure of the dialysis patient, wherein the processing circuit is further to receive a message from the blood pressure device or a microprocessor associated therewith that the dialysis patient is having or has had their blood pressure measured by the blood pressure device; and
in response to receiving the message from the blood pressure device, or the microprocessor associated therewith, the processing circuit is further to send the control signal to the control circuit to begin heating the dialysate.

17. The system of claim 10, wherein the dialysis machine is a hemodialysis machine.

18. The system of claim 10, wherein the predetermined temperature is determined by the processing circuit based on an analysis of medical history data of the dialysis patient, the medical history data including at least body temperature data of the dialysis patient.

19. A control system for a dialysate heater associated with a dialysis machine to pre-heat dialysate used in a dialysis treatment, the control system comprising:
a control circuit operatively associated with the dialysate heater and configured to activate and deactivate the dialysate heater, the dialysate heater configured to heat dialysate for the dialysis machine; and
a processing circuit coupled to memory having executable instructions stored thereon, which when executed by the processing circuit, cause the processing circuit to:
determine whether a trigger event has occurred, wherein whether the trigger event has occurred is determined based on a status of a dialysis patient assigned to the dialysis machine or a status of a dialysis clinic at which the dialysis machine is located; and
in response to the trigger event occurring, send a control signal to the control circuit to activate the dialysate heater and begin heating dialysate for the dialysis machine to a predetermined temperature.

20. The control system of claim 19, wherein the predetermined temperature is determined by the processing circuit based on an analysis of medical history data of the dialysis patient, the medical history data including at least body temperature data of the dialysis patient.
